(19) United States
(12) Patent Application Publication
(10) Pub. No.: US 2003/0203031 A1
(43) Pub. Date: Oct. 30, 2003

(54) Immune modulation with polystyrene sulfonate

(76) Inventor: Kumarpal A. Shah, Searington, NY (US)

Correspondence Address: KEUSEY, TUTUNJIAN & BITETTO, P.C., 14 VANDERVENTER AVENUE, SUITE 128, PORT WASHINGTON, NY, 11050, US

(21) Appl. No.: 10/431,199

(22) Filed: May 7, 2003

Related U.S. Application Data
(63) Continuation of application No. 09/955,803, filed on Sep. 19, 2001.
Application No. 09/955,803 is a continuation-in-part of application No. 09/519,229, filed on Mar. 6, 2000, now abandoned.

(51) Int. Cl. A61K 9/14

(52) U.S. Cl. 424/485

(57) ABSTRACT

Polystyrene sulfonate (PSS) is a known immune-modulation drug for complement inhibition. Our due diligent review of this drug identifies it as a broad-spectrum immune modulation drug that therapeutically impacts innate and adaptive immune functions. The drug is first of a class of drug that modulates immune responses in accordance to recent advances of immunology. The therapeutic applications with relation to HIV are detailed.

A compound for modulating an immune response in medical application in the form of a hydrogel including an ultrapurified polymer, a complement modulator and a gelling agent is also detailed. In a first embodiment the hydrogel has a pore size between 50 kDa and 100 kDa which generally functions as an immune response inhibitor. In a second embodiment, the hydrogel has a pore size greater than 200 kDa which generally functions as an immune response activator. The hydrogel may be applied directly, used to coat foreign materials, or used to encapsulate cellular or non-cellular material.

### CROSS REFERENCE TO RELATED APPLICATIONS

This is a continuation of pending patent application Ser. No. 09/955,803 entitled “Immune Modulation With Polystyrene Sulfonate” filed Sep. 19, 2001, which itself is a continuation-in-part of my pending patent application Ser. No. 09/519,229 entitled “Method for Immune Switching,” filed on Mar. 6, 2000, now abandoned.

### BACKGROUND OF THE INVENTION

1. Field of the Invention

This invention relates to the administration of Polystyrene sulfonate (PSS) as a broad-spectrum immune modulation drug and has potential for wider therapeutic applications. Specifically, the invention relates to encapsulation of an immune modulator such as PSS, wherein the pore size of the capsule acts as an immune switch. Encapsulation technology system and its targeted therapeutic applications are detailed.

2. The Prior Art

Immune related medical problems have caused havoc in medical sciences. The magnitude of challenges posed, for example in HIV/AIDS, is reviewed by Sepkowitz, K. A. (2001) in “Special Article: AIDS—The first Twenty Years” published in The New England Journal of Medicine, 244: 1764-1772. The genesis of “Global HIV/AIDS Crisis” is due to a gathering sense of doom in the face of the scale of global epidemic worldwide. 36.1 millions are infected with HIV; an additional 21.8 millions have died; and 13.2 millions children have become “AIDS orphans”. More than 14,000 new infections occur daily—5.3 million in 2000 alone, including 600,000 in children younger than 15 years old. Approximately 70% of cases occur in sub-Saharan Africa, where in some regions, seroprevalence of HIV among adults exceeds 25 percent. The Caribbean, Southeast Asia, and Eastern Europe are struggling with substantial rates of new infections. The basic details of this killer HIV is provided by Hardy, Jr. W. D. “The Human Immune Deficiency Virus”, Medical Clinics NA, 80:1239-1261. In 2000, the Security Council of the United Nations began to address the possibility that AIDS now threatens the world's security. Urgent attempts are now underway to halt the global spreading of HIV.

1. UNAIDS: Currently they are trying to raise $10 Billion Global AIDS fund (New York Times, Jun. 24, 2001, “The UN Looks at AIDS”).

2. Vaccine therapy is still awaited. In 1997, President Bill Clinton challenged scientists to provide an effective vaccine within 10 years. Among the difficulties confronting researchers are viral heterogeneity, uncertainty about how to achieve optimal immunogenecity, the lack of practical animal models, and the ethical dilemmas involved in conducting primary prevention trials in the United States and abroad.

3. International AIDS Activists: They are demanding three Ds: 1.) The first D is for cheaper, better and safer drugs; 2.) The second D is for more dollars for AIDS research; and 3.) The third D for the Debt pardon of the underdeveloped nations so that more funds could be directed to improve infrastructure of medical facilities (Daily News, Jun. 25, 2001; “UN tackles AIDS drug funding”).

4. Pharmaceutical companies: 100s of new drugs are being researched costing multibillion dollars. At stake are the patent laws and availability of these drugs at cost-effective prices to the consumers (a. “Progress against HIV” Nature Biotechnology (2000) 18:466-467, b. Murphy R. I. (2000) “New Antiretroviral drugs in development, AIDS, S3: 14, S227-234, c. Moore J. P. and Stevenson M. (2000) “New Targets for inhibitors of HIV-1 replicators” Nature Reviews/Molecular Cell Biology, 1: 40-49 and d. Furfine E. S. “The Next Generation of Human Immunodeficiency Virus Protease Inhibitors: Targeting viral resistance, Chapter 4, Proteases as Targets for Therapy,” Edited by Klaus von der Helm, Bruce D. Korant and John C. Cheronis, Springer).

5. HIV Experts:

A. World's leading HIV experts such as Anthony Fauci and Cohen O. at National Institute of Health have opined for the need for better, simple and safer drugs for HIV therapy that target HIV genomic structure in a novel way and at multiple points so that the risk of resistance could be reduced (a. Cohen O. J. and Fauci A. (1999) “Transmission of drug-resistant strains of HIV-1: unfortunate, but inevitable” The Lancet, 354:697-698, b. Yerly S. et al. (1999) “Transmission of antiretroviral-drug resistant HIV-1 variants” Lancet, 354: 729-733).

B. Another leading expert in immunology, Pinto L. A. from National Institute of Health has commented that in view of the limited therapeutic efficacy of current “HAART” regimes, there is urgent need to combine such therapy with immune based approaches that may improve therapeutic efficacy of present cocktail therapies (Pinto L. A. et al. (2000) “Short Analytical Review: Immune-Based Approaches for Control of HIV infection and Viral-Induced Immunopathogenesis, in Clinical Immunology, 97:1-8).

6. Current complexity of HIV management is summed up in the editorial “First Rule of AIDS in Africa: Do No Harm,” in The Wall Street Journal, May 2, 2001.

Despite scientific progress and therapeutic advances—cost ineffective medicines have proliferated and the demand-supply gap for the life saving drugs have widened. This is a true international emergency of the 21^{st} century. Leaders around the world are scrambling to find meaningful solutions. Billions of dollars are raised to address the research and development of new drugs and vaccines. Attempts are underway to streamline intellectual property rights so that life saving drugs can be accessed globally at reasonable costs. Attempts are also underway to accelerate and streamline the drug discovery and drug development process so as to benefit consumers without adversely affecting their safety. Cost effective, meaningful, life-saving solutions that can be applied expediently at a global level are therefore urgently needed.

Central to the understanding of HIV immunopathogenesis is the working of the human defense system. Evolutionary immunologists date Innate or the natural human defense system to 700 million years or more. It is an ancient anti-microbial defense system. Viruses co-evolved with human existence. Viruses are essentially nucleic acid fragments. They are unable to exist on their own. They must find host and use their replication machinery for its own survival. In order to survive in the human host it must develop strategies to confront and defeat human defenses. A central question in the innate immunity is: how are pathogens recognized and how do pathogens thwart the complement system for their own survival?

The complement system is an ancient innate human defense system against microorganisms including viruses. Considerable advances in the working of the complement system have occurred in the last decade. Pangburn M. K. (1998) in “1.2.3 Alternative Pathway: Activation and Regulation” in The Complement System, edited By K. Rother, G. O. Till and G. M. Hansch, Springer Verlag Berlin Heidelberg, 93-115 explains why the Alternative complement system fits the description of the innate complement system more as opposed to specific pathways of classical and mannose-binding protein. The basic workings of the Alternate complement system and its role in the innate host defense system is detailed.

According to “enzymatic tick-over hypothesis” there is spontaneous hydrolysis of the thioester in C3 to generate C3b. The role of C3b in selecting self vs. non-self surface is detailed by Walport M. J. (2001) “Advances in Immunology: Complement: First of two parts,” New England J. of Medicine. 344:1058-1066. Activated thioester bond in C3b enables it to bind covalently to hydroxyl groups or nearby carbohydrate and protein acceptor groups. If the acceptor molecule is a carbohydrate surface of the bacterium, then the enzyme precursor Factor B binds to the C3b to form C3bB. This is susceptible to cleavage and activation by Factor D. This leads to the formation of a C3 convertase enzyme, C3bBb. This activates the complement system to generate C3a to C5a inflammatory intermediate products as well as terminal inflammatory and cytotoxic C5b-C9 membrane attack complex. C3bBb stabilizes by the binding of Properdin. This enzyme cleaves more C3 leading to the deposition of additional C3b on the bacterium.

If the acceptor group is a host cell surface that lacks carbohydrate group, the protective regulatory mechanism comes into play. This is illustrated by the binding of Factor H as C3bH. This is a cofactor to serine esterase Factor I. The action of Factor I depends upon the normal workings of Factor H. Factor I cleaves the C3bH into inactive IC3b. The IC3b no longer can participate in the formation of a C3 convertase enzyme. The carbohydrate environment of the surface on which the C3b is deposited determines the relative affinity of C3b for Factor B. On host cell surfaces bearing polyanions such as sialic acid, Factor H binds with higher affinity than Factor B. On microbial surfaces that lack a polyanionic coating, Factor B binds to C3b with a higher affinity than does Factor H. Pangburn M. K., (2000) in Immunopharmacology, 49:149-157 details the discriminatory power of Factor H to identify self.

Factor H is a complement regulatory protein of the Alternate complement system. It has a unique structure of twenty short consensus repeat (SCR) domain. Each SCR contains approximately 60 amino acids. Factor H is heavily glycosylated and has a high sialic acid content. Factor H is the key self-recognition protein that discriminates host-like features on microorganisms and generates a spectrum of activation rates for the complement system in different microorganisms.

A central question in innate immunity is how its various systems distinguish between potential targets and hosts. Thus, recognition of polyanions is not the function of C3b, but a function of Factor H. Factor H uses its 20 SCR domains to search for and interact with many ligands on a given target. Each SCR domain contributes to the recognition pattern, and if these sites work cooperatively in groups of twos, threes and fours, etc, then by simple combinational math, Factor H would have the ability to discriminate among over 10⁶ target surfaces.

Fearson D. T. and Locksley R. M. (1996) in “The Instructive role of Innate Immunity in the Acquired Immune Responses,” Science, 272:50-54, sets out the fundamental differences in the recognition strategies adapted by innate immunity and adaptive immunity. Innate immunity responses are geared to the recognition of carbohydrate signature in the pathogens. The adaptive immune system is geared to the recognition of peptide fragments that are processed and presented by Antigen presenting cell in the MHC I and II complexes. In adapting to this system, adaptive immunity has essentially lost the innate ability to identify carbohydrate signature. The key circulating or humeral proteins of the Alternate complement system are Factor D and Factor H which regulate the subsequent fate of C3b and its fragmentations into active and inactive particles.

How does HIV circumvent the workings of the complement system and fool it to gain entry inside cells to facilitate its own survival? Stoiber et al. (2001) explains this in “The supportive role of complement in HIV pathogenesis” in Immunological Reviews, 180:168-176. HIV confronts the complement system and activates its functioning. However, it also binds Factor H and masks its identity as Self. The crucial role played by Factor H in protecting HIV is evident if HIV is incubated in Factor H depleted sera. This results in 80% complement-dependant virolysis in the presence of HIV specific Ab's. C3b fragments are utilized by HIV in the human host to gain entry inside CD 4 cells. Therapeutic options such as blocking Ab against CR2 and the use of Pep A-Factor H derived peptide to thwart immune evasion of HIV were suggested.

How is Factor H pirated by pathogens for its own survival advantages? This is detailed by Panguin M. K. 1998 & 2000 as well by German and Finland scientists like Zipfel P. F. et al. (1999) in “Mini Review: Factor H and disease: a complement regulator affects vital body functions,” in Molecular Immunology, 36: 241-248. Factor H is pirated or its function is subverted by a number of bacteria such as Neiseria Gonococci, Streptococcus Pyogenes, viruses such as HIV, and parasites such as Trypanosoma Cruzi. Certain cancers such as cervical, bladder and renal carcinoma secrete Factor H-like substances. Factor H binding with pathogens interferes with C3 convertase flagging of pathogens and it destabilizes the function of C3 convertase. This allows pathogens to escape immune surveillance mechanisms of the host and multiply within the host.

With reference to HIV, where is the binding site for Factor H? This is detailed on its surface protein gp120/41 is studied by an Austrian group of scientists from Innsbruck in collaboration with U.S. based scientists from Utah University. Thus Stoiber H. et al. (1995) reported in “Interaction of several complement proteins with gp 120 and gp 41, the two envelope glycoproteins of HIV-1” in AIDS, 9: 19-26 and by Stoiber et al.(1995) in “Human Complement Proteins C3b, C4b, Factor H and Properdin React with specific sites in gp 120 and gp 41, the Envelope Proteins of HIV-1” in Immunobiology, 193: 98-113. What is the efficiency of Factor H removal in clearing HIV? This question is methodically researched and has been demonstrated. Stoiber et al. (1996) in “Efficient Destruction of Human Immunodeficiency Virus in Human serum by inhibiting the protective Action of Complement Factor H and Decay Accelerating Factor (DAF, CD 55)”, In J. of Exp. Med. 183: 307-310, explains how and why Factor H depletion leads to efficient HIV clearance. The therapeutic significance of this finding is detailed by Dierich et al. (1996) from Innsbruck, Austria explaining how Factor H removal can be used as “A Complement-ary AIDS Vaccine” in Nature Medicine, 2: 153-155 as well as by Stoiber et al. in Immunological Reviews, 2001. Factor H on the surface of HIV inactivates C3 convertase and generates inactive C3 complement fragments. These fragments are used to opsonize HIV particles and gain entry inside CD 4 cells. Speth et al. (1997) from Innsbruck, Austria details this aspect in “Complement receptors in HIV Infection” in Immunological Reviews, 159: 49-67.

PSS has been studied in the basic science literature for immune modulation involving a. Factor D, b. Factor H, and c. CD 4 components of the immunity. It is also studied for its anti-HIV effects. It is also studied for generating vaccine responses.

A. Factor D Inhibition: The role of PSS for complement inhibition has been studied. U.S. Pat. No. 4,265,908 to Conrow et al. describes the complement inhibitory effects of sulfonic salts and its various formulations. The contents of U.S. Pat. No. 4,265,908 is incorporated herein by reference thereto. Several investigators have reported the complement inhibitory properties of PSS. This knowledge has been applied successfully to develop Polysulfone and biosulfane hollow fibers for the therapy of hemodialysis. The successful application of this advances has been reported by a Belgium group of scientists (Vanholder et al. (1994) “Clinical experience with polysulfone: 10 years, in clinical Nephrology, 42: S13-S20). Successful cures of diabetes and Parkinson's disease by cellular xenotransplantations have also been reported by a reputed Japanese group of scientists from Kyoto university (Setoyama H. et al. (1998) “The potential of anticomplement synthetic sulfonic polymers for xenotransplantation”, in Transplantation Proceedings, 30: 67-70; Iwata H. et al. (1999) “Control of complement activities for immunoisolation”, in Annals of the New York Academy of Sciences, 875: 7-23; Date I. et al. (1996) in “Preliminary report of polymer-encapsulated dopamine secreting cell graft into brain”, in Cell Transplantation, 5: S17-S19). Kirschfink M. (2001) summarizes the state of current drugs that are being researched for “Targeting complement in therapy” in Immunological Reviews, 180: 177-189. Geneva and French-based groups of scientists such as Pascual et al. (1993) in “Specific interactions of polystyrene biomaterials with factor D of human complement” studied the mechanism of complement inhibition and reported their findings in Biomaterials, 14: 665-670.

PSS inhibits Factor D which is a complement activating protein of alternate complement pathway. Recent advances of immunology have widened the role and functions of Factor D. Factor D is a serine protease of chymotrypsin family. This family of proteins is involved in the complement pathways involving classical, lectin based and alternate complement system as well as in coagulation cascade. Factor D inhibitors therefore are not specific for Factor D alone but they cross-inhibit classical, lectin based complement pathways and coagulation pathways as shown by USA based group of scientists (Kilpatrick J. H. et al. (1997) “Chapter 13: Control of the Alternate Complement Pathway: Inhibition of Factor D in Controlling the Complement system for novel drug development,” pp.203-225, edited by Mazarakis H. and Swart S. J., Published by International Business Communications, Inc. and Rustagi P. K. et al. (1998) “Development of Novel, Broad spectrum Serine Protease Inhibitors for use as anticoagulants”, Chapter 15, Anticoagulant, Antithrombotic and Thrombolytic therapeutics II, IBC Inc., 307-320). Factor D inhibitors are there for serine protease inhibitors.

B. Factor H inhibition: A curious observation made by Pascual et al. in 1993 was the conflicting finding that the drug PSS binds to Factor H twice stronger than Factor D. What are the mechanisms and kinetics of Factor H inactivation by PSS? Pascual et al., reports the details. At the time these finding were published, the importance of Factor H in immune evasion was not known. These advances came later on, particularly with reference to HIV. Thus, finding that Factor H is inhibited strongly by PSS is a predated medical advance ahead of recent advances of immunology. The immense significance of this finding remains to be explored.

C. CD 4 Inhibition: CD 4 receptors are present on monocytes, macrophages and Antigen Presenting Cells or Dendrocytes of innate immune cells and also on CD 4T cells of adaptive immune cells. These receptors work in conjunction with Chemokine receptors such as CCCR5 and CXCR4 to facilitate HIV entry into CD 4 cells. Complement receptors CR1-CR3 and other receptors such as FcyR may also co-participate in this process. Such intact entry of HIV inside CD 4 cells lead to viral multiplication and the budding out of fresh viruses. This is detailed by Chaplin J. W. (1999) in “HIV Pathogenesis: gp 120-antibody complexes bind CD 4 and kill T4 cells—immunotoxin therapy should prevent the progression of HIV to AIDS” in Medical Hypotheses, S2/2: 133-146; and Hewson T. et al. (1999) in “Review Article: Interactions of HIV-1 with Antigen Presenting Cells, in Immunology and Cell biology, 77: 289-303. What is the role of PSS in inhibiting the surface attachment of HIV? This has been studied independently by Australian group of scientists. Parish et al. (1990) in “The Journal of Immunology,” 145: 1188-1199.

CD 4 cells of innate immunity are involved in processing foreign antigen peptides and lodging them in the grooves of MHC I and MHC II. This provides secondary non-self identity along with expression of co-receptor function such as B7 and adhesion receptors. Dendritic cells essentially act as a transducer between innate and adaptive immunity. CD 4 T and CD 8 T cell initiates adaptive immune responses of vaccine type only if both signals are adequately expressed in CD 4 cells of innate immunity. Failure to express signal 1 and 2 by either drugs or by interference with intracellular processing or destruction of cells by viruses leads to tolerance phenomenon. This prevents the generation of appropriate vaccine responses. This aspect of the immunology is detailed in Bell D. et al. (1999) “Dendritic cells” Advances in Immunology, 72: 255-324; and by Sousa C. R. et al. (1999) in “The role of dendritic cells in the induction and regulation of immunity to microbial infection” Current Opinions in Immunology, 11: 392-399. The recent advances of immunology thus identifies CD 4 cells as being present in both innate cells (monocytes, macrophages and dendritic cells) as well as in adaptive cells (CD 4 T cells). HIV primarily uses CD 4 receptors and the associated co-receptors to gain entry inside immune cells. This is the primary mechanism used by HIV to defeat the expression of secondary non-self-recognition and its associated memory functions as per two signal hypotheses.

Polystyrene sulfonate (PSS) is a drug that stains strongly with Schiff stain (Chaplin A. J. (1999) from the UK reports “The use of histological techniques for the demonstration of ion exchange resins”, in J. Clin. Pathology, 52: 776-779. A Schiff positive drug reacts with CD 4 T cells (T_{H}0) and stimulates T_{H}1 type cytotoxic adaptive immune responses as well as T_{H}2 type neutralizing Ab type B cell responses. (Rhodes J. et al. (1995) from Welcome research group in UK reported “Therapeutic potentiation of the immune system by co stimulatory Schiff-base-forming drug”, in Nature, 377: 71-75; and Higaki M. et al. (1998) from Japan reported “Enhancement of immune response to intranasal influenza HA vaccine by micro particle resin,” Vaccine, 16: 741-745. Pinto L. et al. (2000) from National Institute of health, USA published “Short Analytical Review: Immune based Approaches for control of HIV infection and viral induced immunopathogenesis” in Clinical Immunology, 97: 1-8. In developing immune based therapeutic approaches they commented on the benefit of Flu vaccine having anti-HIV like cytotoxic responses.

PSS has been shown to bind to V3 loop of gp 120, inactivate Reverse transcriptase and Tat enzyme in in-vitro and has been experimented with for its potential to provide STD prophylaxis by U.S. groups of scientists (Lee J. J. in U.S. Pat. No. 5,308,612 entitled “Uses of polystyrene sulfonate and related compounds as inhibitors of Tran activating transcription factor (TAT) and therapeutics for HIV infection and AIDS”; the contents of U.S. Pat. No. 5,308,612 are incorporated herein by reference thereto; Mohan et al. (1992) in “Sulfonic polymers as a new class of human immunodeficiency virus inhibitors” in Antiviral Research, 18: 139-150; Tan G. T. (1993) in “Sulfonic acid polymers are potent inhibitors of HIV-1 induced cytopathogenicity and the reverse transcriptases of both HIV-1 and HIV-2,” Biochemica et Biophysica ACTA, 1181: 182-188; Anderson R. A. et al. (2000) in “Evaluation of Poly (Styrene-4-Sulfonate) as a Preventative Agent for Conception and Sexually Transmitted Diseases” in J. of Andrology, 21: 862-875; and Harold B. et al. (2000) in “Poly (Sodium 4-Styrene Sulfonate): An Effective Candidate Topical Antimicrobial for the Prevention of Sexually Transmitted Diseases,” The J. of Infectious Diseases, 181: 770-773.

Careful perusal of HIV immunopathogenesis suggests that these actions not only have anti-HIV effects, but they essentially target HIV's ability to replicate, mutate and induce immunosuppression in the host (Foucher R. A. M. et al. (1992) from Amsterdam, The Netherlands reported in “Phenotype-Associated Sequence Variation in the Third Variable Domain of the Human Immunodeficiency Virus Type 1 gp 120 molecule,” in J. of Virology, 3183-3187; Zocchi M. R. et al. (1998) where an Italian group of scientists studied “HIV-1 Tat inhibits Human Natural Killer Cell Function by Blocking L-Type Calcium Channels,” in The J. of Immunology, 161: 2938-2943; Cianciaolo G. J. “Chapter 3: Viral Related Proteins in Immune Dysfunction Associated with AIDS” in Human Retroviral infections: Immunological and therapeutic control, edited by Ugen K. E., Bendinelli M. and Friedman H., Kluwer Academic/Plenum Publishers).

In the last decade our knowledge of the complement system has expanded as shown in the articles by Sahu A. and Lambris J. (2001) “Structure and biology of complement protein C3, a connecting link between innate and acquired immunity,” Immunological Reviews 180: 35-48; Song W. C. et al. (2000) “Complement and innate immunity,” Immunopharmacology 49: 187-198. The various pathways and complex interrelationship of the working of the immune system are discussed in Chapter 19 of Anti-microbial Defense Systems in “Biochemical Pathways: An Atlas of Biochemistry and Molecular Biology” edited by Gerhard Michal; a Wiley, J. publication.

Complement system is an ancient anti-microbial system that is also involved in xenotransplants and to a subtle extent in allotransplants. There are several complement-mediated diseases for which there are no effective therapeutic drugs. The deficiency of complement proteins and the failure to generate antibody responses or lytic responses lead to fulminant, life threatening infections. Similarly, piracy of specific complement proteins and/or their receptor functions by certain bacterias, viruses, parasites and cancers leads to immune evasion and opportunistic infections. Wurzner R. (1999) Mini Review: Evasion of pathogens by avoiding recognition or eradication by complement, in part by molecular mimicry, Molecular Immunology 36: 249-260.

The complement system and its components have been given new roles of monitoring self/non-self, and scavenger functions to remove apoptotic cells and immune complexes. The complement system is further involved in the initiation of adaptive immunity and memory responses. Therapeutic impacts of these advances are yet to be realized. It has been recently hypothesized that “the simple antigen migration-localization principle should further our understanding of the events that occur with or without therapeutic intervention in a variety of infectious, neoplastic and autoimmune diseases or after transplantation, and may offer improved rationales for prevention and treatment.” Startzyl T. E. and Zinkernagel R. M. “Review Article: Antigen localization and Migration in Immunity and Tolerance.” The New England Journal of Medicine. Vol.339, No.26, pp.1905-1913.

Encapsulation technology is the packaging, carrier and delivery system for cells and drugs. This technology immunoprotects cells and acts as an extended-release matrix for drugs. This approach is used to develop cures and treatments for many diseases. Several polymers have been used but alginate is particularly favored for sensitive applications such as islet cell encapsulation.

Alginates are commercially available. However, use of these heterogeneous and impure commercial preparations have led to bioincompatible, hemoincompatible and inflammatory reactions with fibrosis, thrombosis, abscess formations, infections, immunorejections and graft failures. This led to a bottleneck in the technology and prevented it from advancement in larger animal models from the 1970 to 1990s. It is to be noted that endotoxin content of commercial alginate preparations was 100,000 E.U./g or greater.

In recent years, purified alginate and other polymers have been introduced commercially. Prokop A. and Wang T. G. (1997) have studied and reviewed the subject in “Purification of Polymers Used for Fabrication of an Immunoisolation Barrier” published in ‘Annals of NY academy of Medicine’, Vol.831: 223-231. Several methods for cell encapsulation have been described in recent years using purified alginate. U.S. Pat. No. 5,459,054 to Skjak-Braek et al. describes a method of encapsulating cells with purified alginate. The material has been used successfully to microencapsulate islet cells to cure type 1 diabetes. U.S. Pat. No. 5,879,709 to Soon-Shiong et al. and U.S. Pat. No. 5,573,934 to Hubell et al. detail methods to further improve biocompatibility of alginate by additional coating with PEG and its applications. U.S. Pat. No. 5,874,099 to Dionne et al. describes applications of purified alginate material in cell transplant technology in general. Leblond F. A. et al. evaluated biocompatibility, hemocompatibility and immune tolerance of alginate in “Studies on Smaller (˜315 micron) Microcapsules: IV. Feasibility and Safety of Intrahepatic Implantation of Small Alginate Poly-L-Lysine Microcapsules”. The findings were published in “Cell Transplantation,” Vol. 8: pp. 327-337(1999). The removal of endotoxin appears to be the critical factor in the above improvements in clinical results.

Endotoxins or Lipopolysaccharides (LPS) activate innate immune responses through CD14 dependent and CD14 independent pathways. The CD14 dependent pathway acts through Human Toll-Like Receptors(HTLRs), while the CD14 independent pathway acts through complement mediated pathways. Both pathways coordinate and translate mechanical and chemical signals through dendritic cells to generate specific adaptive immune responses. Dendritic cells thus essentially act as a transducer. Further, translation of innate immune responses to adaptive immune responses also occur through the breakdown products of C3 complement such as C3b and C3d. Recent advances in endotoxin research suggest a human being is 1,000 times more sensitive to the effects of endotoxin than small animal models. Fetal bovine serum is frequently used in encapsulated technology for cells. It contains lipopolysaccharide binding protein and sensitizes the effects of endotoxins. Bacterial and viral contaminants also act as endotoxin sensitizers. The above factors can also increase endotoxin sensitivity by 1,000 fold or more. The combined effects in human beings therefore could be 1,000 to 1,000,000 fold increased sensitivity to endotoxin. This leads to activation of innate and adaptive immune responses and both causes or contributes to immune rejections, bioincompatibility and graft failure. Outside the capsule it leads to fibrosis and choking of oxygen and nutritional supply, while inside the capsule death of cells occur by apoptotic and nonapoptotic mechanisms.

Complement inhibitors that affect complement pathways at various junctures such as at C1, C3, C5a, C5b-C9 and Factor D have been used with success to control and inhibit endotoxin mediated effects. In experimental models, both the control of endotoxin mediated effects and improved survival have been demonstrated.

U.S. Pat. No. 4,265,908 to Conrow et al. describes the complement inhibitory effects of sulfonic salts and its various formulations for therapeutic uses. Hydrogel formulation of sulfonic salts and its polymer with agarose have been described by Iwata H. et al. (1994) in “Strategy for Developing Microbeads Applicable to Isletxenotransplantation into a Spontaneous Diabetic NOD Mouse” in the J. of Biomedical Materials Research; 28(10):1201-07. Sulfonic polymer has been successfully incorporated to improve biocompatibility in hollow fibers of polysulfone and biosulfane used for hemodialysis. Additionally, sulfonic polymer component has heparin-like properties. Labarre D. J. (1990) reviews this property in “Heparin-like Polymer Surfaces: Control of Coagulation and Complement Activation by Insoluble Functionalized Polymers” in The Int. J. of Artificial Organs. 13:10, 651-657. Pascual M. et al. (1993) details the “Specific Interactions of Polystyrene Biomaterials with Factor D of Human Complement” in Biomaterials. Vol. 14, No. 9: 665-670. Kilpatrick J. M. et al. details “Control of the Alternative Complement Pathway: Inhibition of Factor D,” Chapter 13, pp. 203-225, in controlling the complement system for novel drug development, edited by Mazarakis H. and Swart S. J. published by IBC (1997). Rustagi P. K. et al. details “Development of Novel Broad Spectrum Serine Protease Inhibitors for use as Anticoagulants,” Chapter 15, pp.307-320, in “Anticoagulant, Antithrombotic and Thrombolytic Therapeutics II”, IBC series, 1998. U.S. Pat. No. 5,660,825 to Sims P. et al. describes the composition of polypetide that has complement inhibitory properties and its therapeutic applications. U.S. Pat. No. 5,679,345 to Sanfilippo et al. describes a method for preventing complement dependent rejection of organs or tissue transplants using antibodies which prevent the formation of C5b-C9, membrane attack complex.

As shown by these prior art examples, bioincompatibility, hemoincompatibility and immune rejections are major problems in medicine and surgery including implantation and transplantation biology. Further, it is obvious that the rate-limiting step in the clinical application of foreign materials is the discovery of chemicals and compounds that have inhibitory effects on bioincompatibility and immune reactions. LPS and complement mediated effects are best described in relation to microbial infections. It has been recently hypothesized that “the simple antigen migration-localization principle should further our understanding of the events that occur with or without therapeutic intervention in a variety of infectious, neoplastic, and autoimmune diseases or after transplantation, and may offer improved rationales for prevention and treatment”. Starzl T. E. and Zinkernagel R. M., “Review Article: Antigen Localization and Migration in Immunity and Tolerance,” The New England Journal of Medicine. Vol. 339, No. 26, pp. 1905-1913.

### SUMMARY OF THE INVENTION

It is the object of the invention to provide cost effective, expedient life saving broad-spectrum immune modulation drug such as PSS to address the global needs of HIV/AIDS crisis.

I achieve above object of the invention by due diligent, careful perusal of the prior art literature and discovered the following

a. Polystyrene sulfonate is a new class of broad-spectrum immune modulatory drug that modulate the humeral and cellular functions of both innate and adaptive immune system. The action of PSS on Factor D, Factor H and CD 4 are the predated, serendipitous advances of immunology whose importance is obvious only after advances in immunology are further defined.

b. When the reported actions of PSS are vertically integrated-PSS-As per recent advances of immunology provides complement inhibition, modulate the initiation of adaptive immune responses and can generate effective T_{H}1 and T_{H}2 type vaccine immune responses.

c. In HIV/AIDS the drug provides “Ultra-broad Spectrum Anti-HIV” activity and immune based therapy. It has therapeutic potential to replace current cumbersome “HAART” therapy with better safety profile.

d. In HIV/AIDS the drug has potential to cover all aspects of HIV prophylaxis including Vaccine therapy.

The role of translational research and recent advances of immunology is emphasized in above conclusions.

My ability to solve above problems resides in the facts that from Bench to bedside, from Concept to Clinical application, and from Discovery to dissemination, translating novel scientific insights into new approaches for prevention, diagnosis, and treatment of disease is the ultimate goal of medical research. Advances in medical research results from a series of interrelated and interdependent steps involving basic scientists, applied researchers and clinical investigators. This translational research process includes verification of basic hypotheses and observation with in vitro studies, confirmation in animal experiments and perhaps refinement with biomathametical modeling, and clinical testing in phase 1 to phase three trials (Fontanrosa P. B. and DeAngelis C. D. in “Editorial: Basic Science and Translational research, JAMA, 2001; 285: 2246). Integration of basic science data from diverse global sources conducted by diverse leading scientists with different purposes, in different time zones to solve critical therapeutic problems of our time is a daunting task and requires sharp analytical skills and deep understanding of the medical sciences. Such analysis if done due diligently saves billions in unnecessary experiments and redirects scarce resources to where it is needed most. In recent times the use of Internet and information processing tools have eased these tasks.

a. PSS is an endocrine drug used by me for therapy of electrolyte disturbances such as hyperkalemia since 1975 (a. Gerstman B. B. and Platt R. (1991) “Use of Sodium Polystyrene sulfonate in Sorbitol in the United States, 1985-89” in American J. of Kidney Diseases, Vol. 18 No. 5: 619-620, and b. Sodium Polystyrene sulfonate, Martindale: The Complete Drug Reference, 32^{nd} edition, edited by Parfitt K., Pharmaceutical Press, 1999; 995-996). Ideas and observations from the clinic can be brought to the laboratory or to a more basic level of the translational research hierarchy for further investigation as shown by the new application of this established drug.

b. Drug discovery: This is often a freelance activity pursued at prestigious medical institutions around the world by scientists of repute. This process does not require regulatory approval. PSS is a globally approved drug that has been available in USA since 1975. This drug has several multifaceted properties and therefore is a ready source material for researchers around the world. Several researchers around the world have used this material to pursue their research interests in different medical disciplines. Physicians have the responsibility to remain aware of current advances in biomedical sciences and to understand the application of promising new ideas to clinical medicine. Several immune modulatory actions are reported with PSS but its integration or translation with current advances of immunology was an overlooked component. This has led to underestimating and overlooking of the true significance of PSS. Our due diligent review identifies PSS as a new class of broad-spectrum immune modulating drug that has the capacity to modulate humeral and cellular arms of innate and adaptive immune responses.

c. Drug development process: This is a highly restricted, regulated process that requires addressing safety concerns and efficacy concerns.

Typically, drug regulators require drug sponsor, institutional review board, and clinical trial data in a systemic manner from phase 1 to phase 3 and phase 4 post marketing monitoring of the drug. This is a multimillion dollar undertaking requiring extensive documentations. Only the drug industry is able to undertake or afford such activity. Government institutions and medical scientific establishments in general undertake clinical trials sponsored by drug companies. Their own ability to initiate drug trials is severely handicapped due to regulatory burdens and costs. In certain disease conditions such as immune diseases, surrogate markers are used extensively to judge the efficacy of the drug. Here independent efficacy data by at least two sources provides considerable validity. New applications of established drug is an exception to the drug development process. It provides safety data that normally costs millions of dollars to evaluate prospectively. It provides global availability and ready formulations that require minor modifications. In a deadly disease such as HIV/AIDS where current therapeutic measures fail, use of new untried formulations can be justified in current cases where “HAART” therapy fails. It provides an opportunity to save lives and provide rapid meaningful data that can be improvised for lesser urgent medical conditions. In developing several therapeutic applications related to immune diseases, it might be noted that careful perusal of the data show that clinically approved dosages of the drug are on the average 90 grams/day for hyperkalemia therapy. At these doses, the safety of the drug is evaluated for mucosal applications (oral and rectal) in millions of patients. The dose required for immune applications is less than 1 gram per day as shown by in-vitro and in-vivo results of the drug. This information is directly transferable to therapeutic applications and require minimal burden of proof. Global resources if directed to further develop this drug will provide the most efficient solution to current “Immune related Global Problems”.

It is also an object of the present invention to improve biocompatibility, hemocompatibility and immune tolerance of polymers such as alginate by reducing its endotoxin content to a minimal level.

It is a further object of the present invention to combine ultrapure alginate with an immune modulator for therapeutic purposes.

It is another object of the present invention to control the action and duration of immune modulators by combining ultrapurified alginate and sodium polystyrene sulfonate in a hydrogel formulation.

It is an additional object of the present invention to regulate the pore size of the hydrogel to less than 100 kD to selectively permit inhibition of immune system for targeted therapeutic applications in implantations and transplantation biology.

It is a further object of the present invention to relax the pore size of the hydrogel to more than 200 kD to permit selective activation of the immune response for targeted therapeutic applications in certain infections, cancers and vaccine developments.

It is an additional object of the present invention to characterize drug-device components of the encapsulation technology to develop encapsulation technology system and its targeted therapeutic applications.

It is a further object of the invention to develop an encapsulation technology system that obviates prior art problems and meet or exceed minimal safety criteria of endotoxin content specified by the FDA for drug-devices.

I achieve these above objects of my invention by first developing an Encapsulated Cell Device as detailed in my U.S. Pat. No. 5,976,780. Ultrapurified alginate and sulfonic polymer are combined and cellular material is processed to remove sensitizing factors. This strategy helps prevent the harmful effects of endotoxin.

When 2% alginate with 5% polystyrene sodium are reacted with Ca 100 mM/L, instant hydrogel formulation occurs. Both exchange Na and bind with Ca. This leads to the encapsulation of polystyrene sulfonate in ultrapurified alginate gel. The pore size of the gel obtained is 50-100 kD. Polystyrene sulfonate is a 50 kD polymer but it does not leach out on repeated washing with water or saline and it forms strong, stable bonds with alginate. Factor D, is a 25 kD protein. It readily permeates the gel and avidly binds with polystyrene sulfonate to provide complement inhibition. In the prior art, agarose gel was combined with polystyrene sulfonate. The hydrogel has pores of 10 kD. This would protect NOD mice from immune rejection simply because of its lower molecular cut off as detailed in my U.S. Pat. No. 5,976,780. Complement inhibition could not account for such effects because all complement proteins have molecular weight above 25 kD. Sodium polystyrene sulfonate thus was excluded to perform its complement inhibitory effects. When combined with agarose, to prevent leaching, additional coatings with polybrene and carboxy methylcellulose were required. In sensitive applications such as cell transplant, the procedure of agarose with polystyrene sulfonate requires repeated exposure to thermal trauma. When used intravenously, polystyrene sulfonate acts as a suspension-solution and tends to precipitate out causing unreliable dosing, has a tendency to clump and, due to more avid binding with Factor H, it may activate the alternate complement system. These problems have been obviated when ultrapurified alginate is used in combination with polystyrene sulfonate.

Factor D circulates in the blood in the conc. of 1-2 mcg/ml but is a powerful complement activator. Thus, for example, cobra venom factor will not be able to convert C3 to C3c in the absence of Factor D. However, addition of even 1% of normal concentration of Factor D in-vitro is capable of restoring C3 to C3c conversion. By contrast, bonded Factor D with sulfonic polymer in 100 times greater concentration fails to restore C3 conversion. This indicates total loss of Factor D activity following its binding with sulfonic polymer. The pore size of 50-100 kD prevents binding of Factor H which is a complement inhibitor. Factor H, as a complement inhibitor, accentuates the degradation of C3 convertase, binds with breakdown products of C3 convertase and prevents costimulation of B cells and work with Decay Accelerating Factor (DAF) and Factor I to rapidly inactivate C3 convertase. Its binding with polystyrene sulfonate and its inactivation of complement inhibitory property, is therefore not desirable. Selective binding of Factor D and selective exclusion of Factor H may help potentiate complement inhibition in a 50-100 kD pore size gel. The strategy helps improve reliability of sulfonic polymer as a complement inhibitor. By preemptive blockage of the complement system, transducer function of dendritic cells is not activated. This way, complement inhibitors also act as an inhibitor of adaptive immune responses.

Encapsulation of sodium polystyrene sodium in 200 kD pore size alginate will have opposite effects viz. activation of complement system by more avid binding with Factor H. This in turn will lead to activation of transducer function of dendritic cells. Thus, complement activation also contributes to activation of adaptive immune responses. The relaxation of pore size can readily be obtained by using appropriate dilution of alginate and CaCl concentration and by shortening the reaction time. The activation of immune response is beneficial in vaccine development as an adjuvant to speed immune responses. Thus, it can be used as military or civilian preparedness against bioterrorism. Certain bacterial infections, viral infections, parasitic infections and cancers evade immune system and thrive by binding with Factor H or by secreting Factor H-like substances. Streptococcus pyogenes, Neisseria gonorrhoeae, HIV, Echincoccus granulosus and Yersinia entercolitica are such examples of infections that evade immune responses by binding to Factor H. Cancers, such as of cervical, bladder and renal cells, evade immune response by secreting Factor H-like substances. Use of Factor H antibodies has been shown to bring efficient killing of resistant strains of N. Gonorrhea and HIV in in-vitro experiments. It is speculated that encapsulation of sodium polystyrene sulfonate in 200 kD pore size polymer will allow direct binding of Factor H and deplete its availability for pathogens and tumors. Thus, it will transiently activate the complement system. Concomitant administration of antibiotics, antivirals or antitumor agents will therefore be more effective in eradicating such pathogens or tumors.

Sodium polystyrene sulfonate, when administered intravenously in experimental animals, in doses of 10 mg/kg or more, completely inhibits complement pathways. Further as Factor D inhibitor, it has broad spectrum inhibitory effects on serine protease of chymotrypsin family. This property has protected death of mice from endotoxinemia. Ultrapurified alginate and polystyrene sulfonate act synergistically to inhibit proinflammatory cytokines and complement system. By depriving survival stimuli, such as endotoxin, C3b, C3d and cytokines, antiapoptotic proteins of the monocytes and Bcl family are not activated. The strategy breaks the critical linkage, i.e. breakdown products of C3 convertase and activation of monocyte/dendrocytes to initiate adaptive immunity. This helps prevent activation of adaptive immunity as well. Dendritic cells are not activated and therefore do not express second signal. This leads to arrest in T cell mediated adaptive responses. Lymphocytes and monocytes die “by neglect or passive death.” Cytokines and complement mediated signals are also needed to upregulate adhesion receptors and for cellular activation and aggregation. Failure to provide such signal, leads to nonthrombotic endothelial surface and noninflammatory state. Different hydrogel formulations can readily be prepared using commercially available devices for microencapsulation and hollow fibers for macroencapsulation. Such devices permit targeted therapeutic applications in-vitro, in-vivo or ex-vivo. In-vivo applications could be both extravascular or intravascular.

### BRIEF DESCRIPTION OF THE DRAWINGS

In the accompanying drawings to which reference is made in the instant specifications which is to be read in conjunction therewith, like reference numerals are used to indicate the parts in the various views:

FIG. 1 is a flowchart illustrating the Alternate Complement System and its vertical integration with recent advances of immunology.

FIG. 2 is a flowchart illustrating the short-circuiting of immune responses by HIV and its piracy of Factor H.

FIG. 3 illustrates the life cycle of HIV in the host and how PSS provides ultra-broad spectrum anti-HIV effects and immune-modulation.

FIG. 4 shows endotoxin or LPS effects on afferent and efferent arm of immune responses.

FIG. 5 compares the endotoxin content of a purified alginate with ultrapurified alginate.

FIG. 6 is the immune switch according to an aspect of the invention.

### DESCRIPTION OF PREFERRED EMBODIMENTS

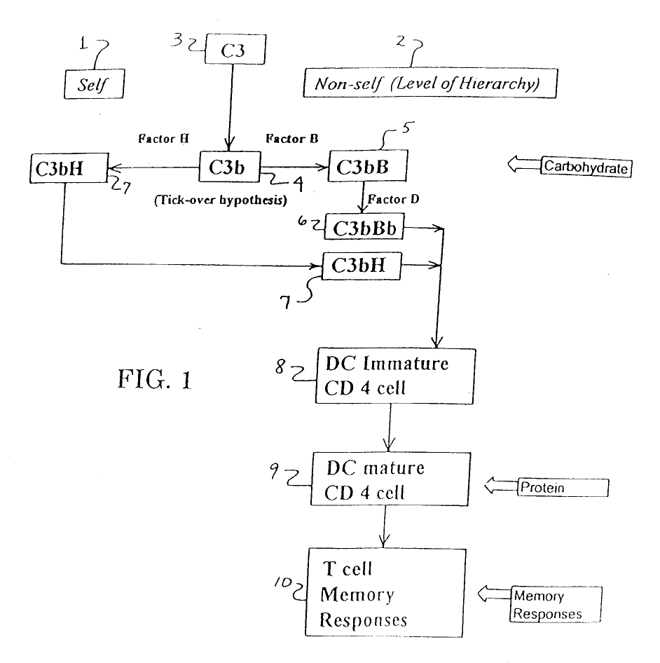

As shown in the FIG. 1, the innate complement system differentiates self, 1 and non-self, 2 through C3, 3 and its active catalysed product, C3b, 4. Identification of polyanionic surface on host surface by Factor H, lead to formation of C3bH, 7. In the absence of Non-self no further C3 convertase is formed. C3b is constantly generated due to “enzyme tick-over hypothesis”. If Non-self or bacteria is identified because of carbohydrate signature it carries, C3b, 4 bind preferably to Factor B to generate C3bB, 5. This is further acted upon by Factor D to generate C3 convertase, C3bBb, 6. C3bH, 7 now has a regulatory function. Active C3 fragmented products, C3a-C5a and C5b-C9, membrane attack complex have inflammatory components. C5b-C9 in addition has cytotoxic component that form membrane attack complex and cause cytolysis. The active C3 fragments contribute to CD 4 or dendritic cell, 8 by expression of co receptor function such as B 7 molecule. Inactive C3 fragments generated by C3bH, 7 contribute to CD 4, dendritic cell, 8 for phagocytosis response of fragmented antigens. CD 4, dendritic cell, 8 processes antigen fragments inside cell and lodge them in the groove of MHC I and MHC II. This allows CD 4 cells, dendritic cell, 9 to mature. T cell, 10 identify secondary Non-self identity in accordance to “two signal hypothesis”. This means, both signal 1 of MHC I and II and signal 2 of B7 co receptor function must be expressed. If they are not expressed, CD 4 T cells, will not sense the presence of Non-self and no clonal or memory responses are generated.

The human defense system is much like a military defense organization of the country by analogy. Factor H is the controlling element that acts as radar and scans for self and determines the primary identity of self. This is done by identifying polyanionic or sialic acid binding sites as self. Factor D identifies non-self. The primary identity of non-self by the innate immunity depends upon its ability to recognize carbohydrate signature. Factor D (25 KD) is the complement protein of the alternate defense system that has the smallest molecular weight. It is ubiquitously present through out the body and therefore a convenient molecule to sense the non-self as primary danger signal. Upon encountering non-self, it triggers a chemical alarm system in the form of activation and amplification of the alternate complement system. The function of activated fragments (C3a, C4a and C5a) and amplified alternate complement system (C5b-C9) is to localize the non-self antigen, produce inflammatory responses around it, mobilize the army of leucocytes and CD 4 containing cells (monocytes, macrophages and dendritic cells) and remove the non-self antigen expediently. Factor H besides identifying self controls the degree of alarm function by generating inactive C3 fragments. C3 convertase along with inactive C3 fragments flag the nonself; opsonize it for phagocytosis by CD 4 cells. CD 4 cells that are involved in the phagocytosis and expression of secondary Non-self expression are immature dendritic cells. Inactive C3 fragments are also involved in stimulating B cell function and act as its co-receptors. CD 4 cells process the non-self antigen inside its cellular machinery and flag its peptide fragments in MHC I and MHC II groove. The active C3 fragments also influence CD 4 cells to express co receptor such as B7 and adhesion receptors. Once secondary expression of Non-self is complete, CD 4 cells are now called as matured dendritic cells. CD 4 T cells and CD 8 T cells sense the presence of non-self peptide fragments only in the presence of associated co receptors such as B7. Thus expression of two foreign signals in denditic cell provides the secondary identity of non-self. T cells sense the secondary non-identity of dendritic cell only if two signals are expressed. The identity of self is maintained if dendritic cells are not matured as in normal state, or if dendritic cell maturation of two signals is prevented or subverted either by drugs or by pathogens such as viruses. The secondary identity of non-self provides back up responses in the form of clonal expansion and mobilization of T cell responses in the form of neutralizing Ab and cytotoxic cells to further assist human defense system for the removal of non-self. Co receptor function imparts memory function to T cells. In the case of repeat invasion by non-self, back up reserve force is directly activated to deal with the intrusions. The vertical integration of Factor H, D and CD 4 function in FIG. 1 is thus in accordance to recent advances of immunology where Non-self identity has three levels of hierarchy (1-3). Each level must proceed sequentially to be fully effective.

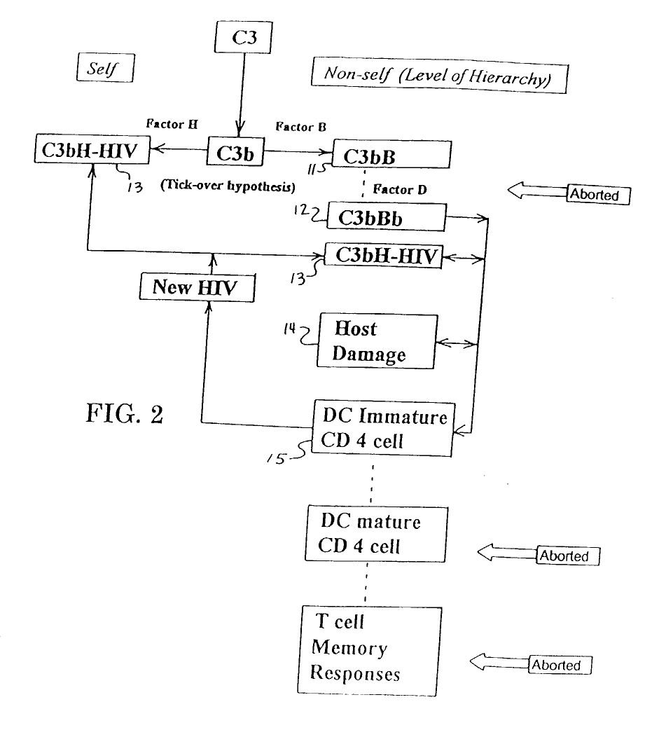

As shown in the FIG. 2, HIV as an intruder to human defense system encounters complement system. It is an enveloped virus and has allo MHC 1 and MHC 2 proteins of the primary host and its complement regulatory machinery on its surface. However, it is sensed efficiently by the complement system as an intruder and alarm system is triggered. HIV simultaneously binds to host Factor H to form C3bH-HIV, 13. This is similar to hijacking of the controlling element of the defense system. Recruited CD 4 cells at inflammatory sites no longer can identify or locate the intruder or non-self. HIV thus masks the primary identity of non-self. C3 flagging of non-self HIV do not occur and complement system though activated (C3a, C4a and C5a) it does not proceed to C5b-C9 to generate cytotoxic responses (1-aborted). HIV by hyper-activating alarm system, C3bBb, 12 inflicts inflammatory damage to the host, 14. Activated C3 fragments are now inactivated by Factor H hijacked by HIV. Inactive C3 fragments are used to gain entry inside CD 4 cells, 15. Human defense system fails to identify intruder as non-self, it fails to flag intruder and fails to efficiently fragment HIV. HIV now exploits complement fragments and receptor machinery of cells for successful entry inside CD 4 cells or immatured dendritic cell, 15. The very cells that suppose to phagocytose HIV and present its peptide fragments to MHC 1 and 2 now supports the multiplication of HIV. This aborts secondary Non-Self identity (2-aborted), 15. HIV multiplication generates HIV proteins that further cause immunosuppression and aborts expression of memory responses. HIV thus masks the secondary identity of non-self by preventing dendritic cell maturation and subverting its function for its own survival and replication. Masking of secondary identity of non-self prevents generation of secondary defense forces in the form of neutralizing Ab and cytotoxic cells. The New HIV budding out and repeats the cycle of CD 4 entry to systemically destroy its immune function and pave the way for AIDS by short-circuiting the functioning of the human defense system, 16.

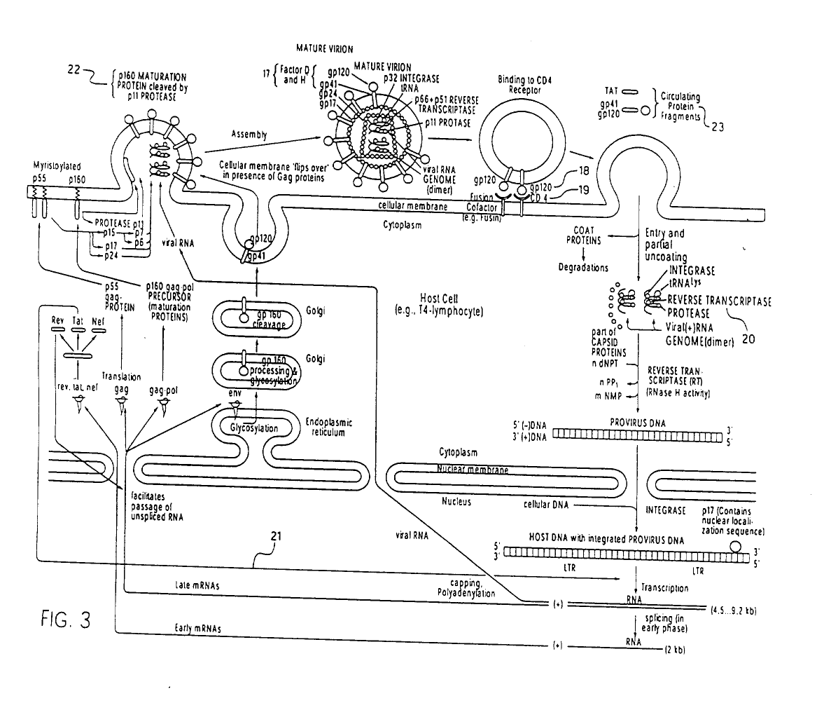

As shown in FIG. 3, PSS is a polyanionic, foreign biomaterial that mimics the sialic acid or polyanionic properties of host cells. It competes with HIV for Factor H, 17, Factor D, 17 and CD 4 cell surface, 18-19. This results in inhibition of Factor D, Factor H and surface attachment of HIV to CD 4 cells, 17-19. In addition, HIV proteins such as reverse transcriptase (RT), 20 is inhibited outside the cells. Similarly, Tat protein, 21 is inhibited. Serine proteases are involved in gp120 maturation. PSS, being a serine protease inhibitor is expected to inhibit 22. HIV proteins are inhibited mainly outside cells, 23 that are involved in immunosuppression Thus PSS is a new class of broad immune modulation drug that can affect the humoral and cellular function of innate and adaptive immune system and provide ultra-broad spectrum anti-HIV effects.

Table 1, details the action of PSS and its intended immune modifying effects on host as well as HIV.

TABLE 1
PSS effects: Therapeutic Immune responses

| NO. | Immune component | PSS Actions | Effects |
|---|---|---|---|
| 1 | Innate (Humoral) | Inhibition *2 | 1. HIV exposed to circulating Ab |
| | Factor H | | 2. HIV killed efficiently by circulating Ab |
| | | | HIV fragments processed by CD 4 cells for |
| | | | secondary level of Non-self hierarchy. |
| 2 | Innate (Humoral) | Inhibition *1 | 1. Reduce host inflammation |
| | Factor D | | 2. Pathway proceed to C5b-C9 level |
| 3 | Innate and Adaptive | Surface inhibition | 1. Reduce CD 4 entry |
| | (cellular) | | 2. Reduce CD 4 replication |
| | CD 4 (Dendritic cells, | | 3. Reduce CD 4 HIV proteins secretions |
| | Immature) | | 4. Reduce immunosuppression |
| | | | 5. Reduce CD 4 destruction |
| 4 | HIV proteins | Inhibition | 1. Reduce Replication (RT, Tat) |
| | (V3 loop of gp 120, | | 2. Reduce Mutational change (V3, RT) |
| | Reverse Transcriptase | | 3. Reduce immunosuppression (gp120, Tat) |
| | (RT), Tat | | |
| 5. | Innate and adaptive | Stimulated | a. Neutralizing Ab responses |
| | immune cells | i. Adjuvant | b. Cytotoxic responses |
| | CD 4—CD 4 T cells | effect for Th2 | (Vaccine like effects) |
| | Interactions | ii. Schiff positive | |
| | | Th1 responses. | |

My U.S. Pat. No. 5,976,780 titled “Encapsulated Cell Device” details the formulation strategy to combine PSS with ultra-purified biomaterial that reduce nonspecific or background immune activation and meets the endotoxin content criteria's as recommended by FDA. The contents of U.S. Pat. No. 5,976,780 are incorporated herein by reference thereto. The immunoisolation strategy allows selective control of pore size to regulate the entry of desired immune proteins and can be used to develop drug devices to meet disease specific requirements for blood contacting applications.

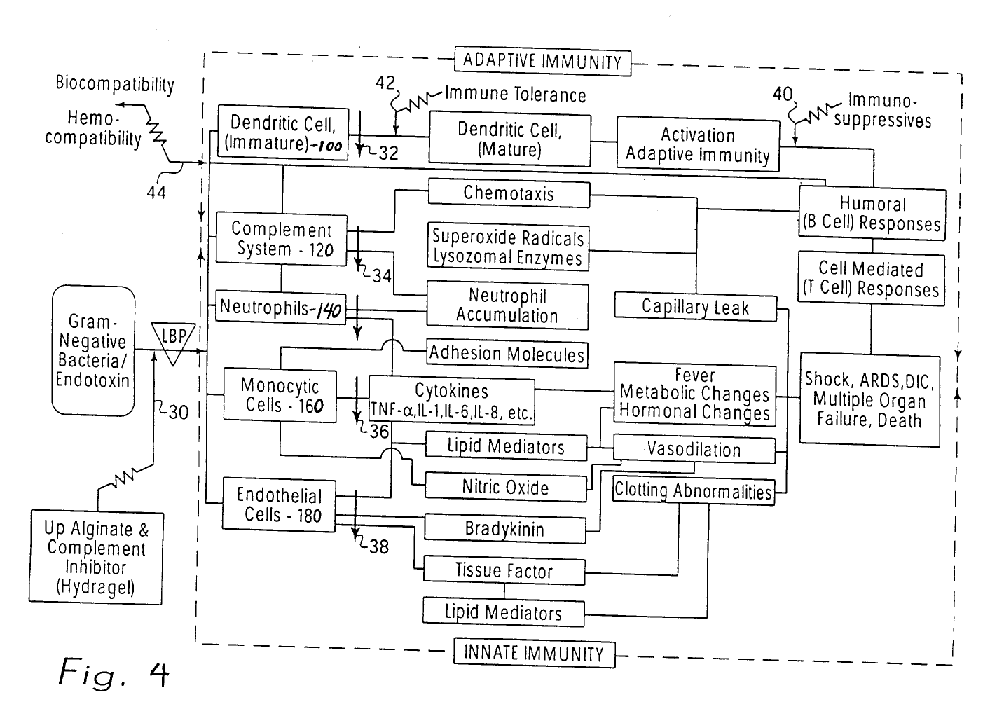

As shown in FIG. 4, endotoxin causes activation and amplification of effector or efferent arms of immune responses through its binding with LBP and complement. The items in the boxes 100, 120, 140, 160 and 180 show the activation of cellular pathways leading to activation of cytokines, costimuli and adhesion receptors. The end result is bioincompatibility, hemoincompatibility and immune rejections. In a worst case scenario, septic shock and death may occur. Removal of endotoxin to its minimal level thus prevents the activation of items 100, 120, 140, 160 and 180. Blockage of cellular pathways 32, 34, 36, and 38 occurs and contributes to biocompatibility, hemocompatibility and immune tolerance as shown 44.

Endotoxins contribute to activation of adaptive immunity through C3 breakdown products and monocyte/dendritic cell activation 100 and activation of dendritic cells leads to expression of costimulli, such as B7-1 and B7-2. Costimulation of B7-1 and B7-2 are the critical events that lead to migration of dendritic cells to secondary lymphoid tissues and activate adaptive immunity. For the activation of CD 4+T mediated humoral immune responses, CD+8 T cell mediated immune responses and B cell mediated antibody responses, at least two signals are needed. In the absence of second signal or B7 costimuli, T and B cells fail to cause immune responses and lead to anergy. Prevention of dendritic cell maturation therefore is the critical step to induce immune tolerance 42 to foreign materials including cell and organ transplantations. This occurs by blockage 32 of dendritic cells and its maturation.

Majority of immunosuppressives such as cyclosporine, tacrolimus (FK 506), Azathioprine, Mycophenolatemofetil(CellCept), MuromonaCD3 (OKT3;Orthobiotech), Interleukin-2 receptor antagonist (Basiliximab(Simulect, Novartis) and Daclizumab(Zenapax, Hoffmann-La-Roche) and antibodies to T cell or its costimuli used for induction protocol are targeted to inhibit T cell mediated effector or efferent immune responses 40 to cause immunosuppression after the expression of costimuli or after the activation of immune system or maturation of dendritic cell. Removal of endotoxin, dampening of inflammatory cytokines and complement inhibition provide preemptive strategy to prevent activation of immune responses and lead toward immune tolerance in cell and organ transplant. Presence of memory clone of T and B cells may still activate the immune system. However, in the absence of costimuli the response tends to be milder. Hydrogel nature of the formulation allows one to combine any immuno-suppressives to further strengthen immuno-suppressive effect 40 to cause immunosuppression. Complement inhibitor as a hydrogel formulation can be used as a drug additionally to inhibit complement mediated events including whole body inflammatory responses and septic shock. Hydrogel formulation permits encapsulation of any cell to further protect from immunorejections.

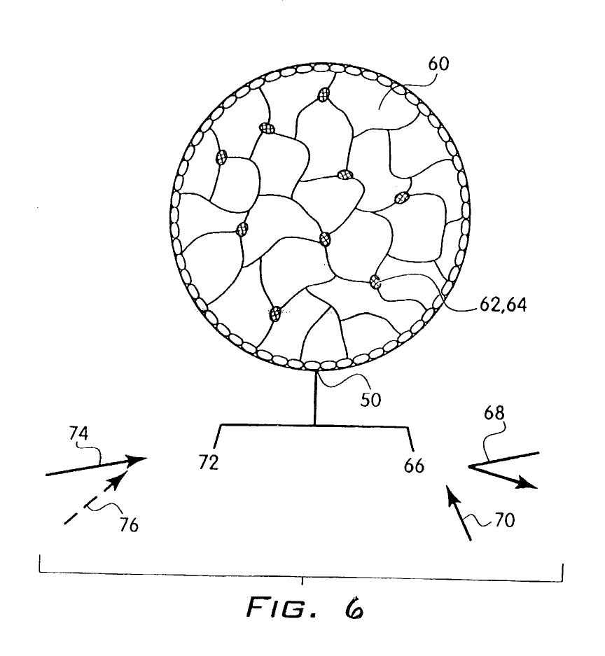

By enlarging pores of the hydrogel to 200 kD, the encapsulated drug acts as an immune activator that has a range of applications in infections, cancers and vaccine developments. As shown in FIG. 6, by controlling the pore size the immune switch function is regulated. Since any drug can be encapsulated, the immune switch function and its potency can be controlled for target specific applications. The micro hydrogel 60 according to the invention contains polystrene sulfonate 62 Factor D and Factor H inhibitor 64. Hydrogel 60 includes pores 50 which, on the one hand, may have a pore size 66 having a nominal molecular weight cutoff between 50 kd and 100 kd. Such a pore size would exclude Factor H, as illustrated by arrow 68, and would allow selective entry of Factor D, as illustrated by arrow 70. This results in immune inhibition. Alternatively, pores 50 may be of a pore size 72 having a nominal molecular weight cutoff greater than 200 kD. Pore size 72 would allow preferential entry of Factor H due to more avid and rapid binding of Factor H as compared to Factor D, as illustrated by arrow 74. Arrow 76 indicates delayed entry of Factor D.

Encapsulation Technology System and Its Targeted Therapeutic Applications

Recently, the FDA has defined encapsulation technology as ‘Therapeutic Drug-Device.’Drug components may be cellular or noncellular and will vary depending upon the targeted therapeutic application. The invention herein provides a device component which may be a capsule, syringe, microcapsule, macrocapsule or ex-vivo instrument. It has the same variability as a drug component. A matrix component (i.e. alginate), however, is the common component and its function is to provide biocompatibility, hemocompatibility and immunetolerance. Addition of gelling agent, such as CaCl, imparts the immunoisolatory and extended-release functions to the matrix. Thus standardization of the two common components, viz. matrix and gelling agent, in the form of a kit permits the development of targeted therapeutic applications using different devices and cellular or noncellular therapeutic agents. Encapsulation of an immune modulator broadens the range of targeted therapeutic applications in implantations, transplantations, certain infections, cancers and vaccines. Since immune modulator is a sulfonic polymer, addition of sulfonic group improves basic biocompatibility, hemocompatibility and immunotolerance of the encapsulated device. Use of ultrapurified alginate of high ‘G’ monomer composition, removal of divalent toxins and presence of sulfonic group contribute to increased mechanical strength and salt bridge stability over a wide pH range. In other words, encapsulation of an immune modulator such as sulfonic polymer results in the development of an encapsulation technology system that overcomes prior art problems and provides a wide range of targeted therapeutic applications.

Described below are methods to formulate encapsulation devices of different shapes and sizes that allow targeted therapeutic applications.

1. Drug Formulation: Alginate is the most common polymer used for the encapsulating cellular and noncellular materials. Below, basic alginate formulation is described that meets or exceeds FDA acceptance criteria for minimal endotoxin content for encapsulated devices. Such formulation is standardized for its chemical properties as detailed in my U.S. Pat. No. 5,976,780.

Step 1. Obtain UP alginate from sources as powder.

EX. UP MVG (S. NO. Property Product Code: Ultrapurified 28023316).

Endotoxin content 700 E.U./g dry weight.

Step 2. Prepare 2% solution of defined quantity in 0.9% NaCl solution.

Endotoxin content in 2% 100 ml solution will be 1400 E.U./dl or 14 EU/ml.

Assuming 0.9% NaCl is endotoxin free.

Comment: Alginate powder dissolves poorly and tends to clump. Magnetic shaker and vigorous shaking are required to adequately dissolve alginate powder. It may take up to 24 hours to completely dissolve alginate powder in the solution.

Step 3. Add appropriate preservative or antibiotics to prevent bacterial and fungal contamination and improve shelf life.

Step 4. Adjust pH to 7.4. Sterile filter solution through 0.45 micron filter.

Step 5. Add dry or wetted PROSEP*-Rem Tox to above solution. Add 1 gm for every 10 ml solution.

Step 6: Gently mix the suspension for 3 hrs. The mixing can be carried out by placing the container on a roller-mixer. Do not use magnetic stirrer at this stage. This can cause break-up of glass beads.

Step 7. Remove the superpatent by allowing the PROSEP* Rem Tox to settle and decant the solution. Alternately a glass sintered funnel can be used to separate the beads from the solution. For 2% 100 ml solution, endotoxin content will be reduced from 1400 EU/dl to 14 EU/dl.

Step 8. Supply alginate solution is 2% ready-to-use sterile solution in vials of 50 ml size. Each vial will have endotoxin content of 7 E.U.

The above ready-to-use polymer solution should be further characterized for microbial growth and shelf life. Appropriate preservatives may be added. Prior to use, alginate solution is diluted to 1% for cellular or noncellular applications by using 0.9% NaCl. Further pH adjustments are not necessary. For most cellular and noncellular applications, requirements of alginate are 5 ml to 100 ml. 1% ml alginate solution contains 7 E.U. of endotoxin. FDA requirement of endotoxin content limit is 5 E.U./kg i.e. 350 EU/70 kg or 20 EU/device. Above procedure meets and exceeds such requirements. In addition to meeting FDA criteria of minimal endotoxin content, hydrogel formulations should be mechanically stable and should not expand or contract or crack or alter its pore size following implantation. Chemical composition of UP Alginate as defined in U.S. Pat. No. 5,976,780 is free of divalent toxins and rich in ‘G’ polymer content. Isotonic gel in 0.9% NaCl, will lead to mechanically stable hydrogel and will maintain its defined pore size. Addition of a sulfonic group further increases salt bridge stability over wider pH range.

Above solution now can readily be used for encapsulating cellular as well as noncellular material. Additionally, the solution can be used to develop target specific therapeutic applications. Thus, for microencapsulation, macroencapsulation or ex-vivo blood contacting applications, commercial devices such as INNOVA microencapsulator or A/G technology hollow fibers and cartridges can readily be used. Use of adaptable nozzle permit one to obtain preselected size and provide protocol flexibility. Further sonification or fragmentation of microcapsule lead to formation of ultramicrocapsules. Less than 5 micron size particles can safely be administered due to its hemocompatibility. The mean size of capillaries is 8 micron and the mean size of RBC is 5 micron. Therefore, particles less than 5 micron will not lead to vascular obstruction. Encapsulation of sulfonic polymer in 50-100 kDa pore size will allow prevention of ischemia-reperfusion injuries. Porous nature of the gel will allow oxygen diffusion to readily take place without resulting in hypoxic damage. This allows ultramicrocapsules to ferry blood and provide extend-release matrix for intravascular drugs such as antibiotics.

### EXAMPLES

### Targeted Applications

1. Improvement in Biocompatibility, Hemocompatibility and Immune Tolerance of Foreign Materials:

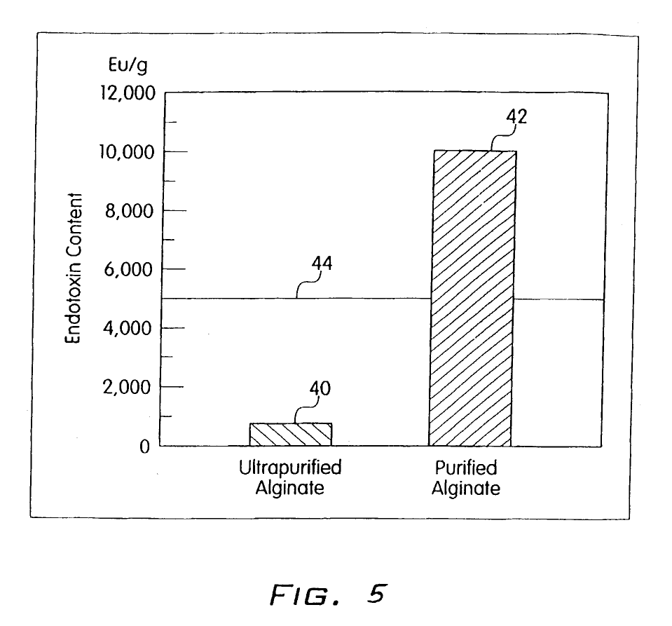

Hydrogel formation of ultrapurified alginate and polystyrene sulfonate form the basic template to improve biocompatibility, hemocompatibility and immune tolerance. A comparison of the endoxtoxin content of ultrapurified alginate 40 with purified alginate 42 is illustrated in FIG. 5. This template can be used in abdomen, pelvic and spinal surgery to prevent postoperative adhesions. In burns it can serve as dressing material. Further it can be used as biocot to improve biocompatibility of foreign materials including other cell transplant procedures. Basic procedure in all these examples is to coat the surface with 2% ultrapurified alginate with or without polystrene sulfonate and developing 50-100 kD pore size hydrogel by spraying the solution with 1-2% CaCl. In blood contacting surface prosthetic devices containing ultrapurified alginate with polystyrene sulfonate can be devised. CaCl in 1-2% added to form 50-100 kDa pore size. This will lead to improved hemocompatibility, biocompatibility and immune tolerance in a variety of blood contacting surfaces such as hemodialysis equipments, cardiopulmonary bypass circuits, etc. In cell transplants, such as endocrine, neural, hepatic cells and somatic gene therapy, there are prior art problems that have defied appropriate solutions and have lead to immune rejections and graft failures. Ultrapurified alginate with complement inhibitor can be used to prevent such occurrences with any foreign materials.

The strategy permits relaxation of pore control to 100 kD. This may be crucial for hepatic cell transplant for the egress of various proteins synthesized. Further, in critical sites, cells can be directly mixed with alginate+complement inhibitor and can be co-injected with a gelling agent such as CaCl₂ in a dual lumen syringe. Such a strategy is of value for example, in neural transplants. The need for precise pore control can be obviated due to immunoprivileged site, removal of endoxtoxin in the alginate and addition of complement inhibitor. The same approach is also applicable to bring world-wide cure of diabetes by incorporating islet cells. In fact any cellular or noncellular therapy can be achieved with this type of encapsulation technology system. FIG. 6 illustrates the components within such microhydrogel 60 contains one or more complement inhibitors—for example, sulfonic polymer like polystyrene sulfonate 62 or any other Factor D inhibitor 64. The alginate hydrogel 60 has pore sizes 66 between 50 and 100 kilo-Daltons(kD) which excludes large molecules—for example Factor H 68 which has a molecular size of 150 kD.

2. Inhibition of Innate Responses:

a. Septic Shock.

Septic shock is a preterminal event with high mortality. It occurs following gram-negative bacterial infections in the setting of major injuries, organ failure and extremely stressful situations in elderly subjects. Endotoxin causes antiapoptosis of hemopoietic cells while paraenchymal cells undergo apoptotic changes leading to organ failures, hypotension and poor tissue perfusion lead to complement mediated events. Widespread activation of serine proteases of chymotrypsin family occur. Multiple treatment approaches are required. Hydration, resuscitation measures, use of antibiotics are common. Ultrapurified alginate with polystyrene sulfonate may be combined with therapeutic doses of antibiotic, antiapoptic substances such as Hepatocyte growth factor and may be administered systematically to inhibit innate responses. Additionally, oral therapy of encapsulation of activated charcoal and “Prosep-RemTox” may be carried out to remove endotoxins from G.I. tract. Similar measures may also be employed prophylactically prior to onset of septic shock to remove endotoxin by using a prosthetic device containing encapsulated activated charcoal and “Prosep-RemTox”.

b. Prevention of ischemia-reperfusion injuries: Ischemia of small blood vessels and its reperfusion leads to activation of complement pathways and thrombosis. This can occur in hypovolummic conditions after surgery, following coronary angioplasty or bypass surgery; in transplant of organs at the time of organ harvesting, rewarming phase or immediately after reperfusion phase of transplanted organs. Hydrogel formulation of ultrapurified alginate with complement inhibitor is expected to have protective effects in preventing thrombosis of vessels.

c. Xenotransplants: Endothelial surface of the transplanted organ is highly reactive. It binds with naturally circulating antibodies to xenoantigens. Endothelial activation provides binding site for classical components such as C1q to initiate complement cascade. Upregulation of adhesion receptors, inflammatory cytokines and thrombotic responses may occur. Use of hydrogel formulation of ultrapurified alginate and polystyrene sulfonate will down regulate complement cascade and help prolong organ viability and its function.

3. Inhibition of Adaptive Immunity.

a. In skin transplants: The wound or surface area where skin is to be grafted is covered with ultrapurified alginate with 5% polystyrene sulfonate. This is then sprayed with 2% CaCl₂. This will lead to instant gelling. The skin is grafted on top of this and sutured at the edges. Additionally, dressing is applied to prevent slippage of the graft. The hydrogel acts as an immunoisolation device and down regulates innate and adaptive immune responses. By preemptively blocking inflammatory cytokines, costimulli and inhibition of complements, dendritic cells will not be allowed to mature. This will lead to immune tolerance. Growth factors, immunosuppressives or factors such as interleukin 100 that prevent dendritic cell maturation may be added as desired.

b. In organ transplants: It has been shown that 300 micron hydrogel containing islets are well tolerated by endothelial linings of liver. It is biocompatible, hemocompatible and does not lead to immunorejections. Porous nature of hydrogel allows oxygen diffusion to take place and prevents ischemic damage to the organ. A critical difference between cell and organ transplants is that in cell transplants the microcirculatory unit is disrupted, while in organ transplants this is preserved. In both cell and organ transplants, endothelial cells, as well as donor dendrocyte or antigen-presenting cells (APC), cause MHC class II restricted immune reactions. However, in organ transplants, this results in endothelial blockage, ischemia and organ failure. In transplanted organs, the oxygen supply to cells is provided by the microcirculatory unit in accordance with Fick's law of gaseous diffusion, while this is not the case in cell transplants. It is reasoned, therefore, that endothelial gelling will not interfere with oxygen supply and may help protect against ischemia-reperfusion injuries or organ transplant rejections. This concept can be extended to transplanted liver, for example. A double lumen catheter can be placed in the portal vein. Ultrapurified alginate and polystyrene sulfonate can be injected through one lumen of the catheter. Other lumen is used for injection of CaCl₂ to form a hydrogel in the endothelial capillaries and then followed with 2% CaCl. This will protect endothelial lining and at the same time provide immunoisolation to the liver thus preventing dendritic cell activation. This strategy is needed to avoid embolic escape of gelled material. Like in skin transplants, hydrogel can be enriched with interleukin 100, TGF-B, hepatocyte growth factor or immunosuppressives or can be combined with bone marrow chimerism for better immunotolerance and graft acceptance. Site specific delivery of growth factors, drugs and cells could be done using this approach.

4. Miscellaneous Applications.

a. Isolation of cells from organs: Enzymes such as collaginase can be immobilized, for example, in pancreas isolation to selectively isolate islet cells and avoid the need for cooling of isolated cells. Collaginase enzyme is the most costly component used in cell isolation procedure. Currently there is no approach described for the economical handling of this enzyme. During islet cell isolation, collaginase enzyme can be mixed with ultrapurified alginate with polystyrene sulfonate and injected inside the pancreatic duct. CaCl₂ can be co-injected simultaneously or sequentially to gel entrap collaginase enzyme. In the presence of Ca ions, collaginase activity is increased during warming phase. Its leakage in surrounding bathfluid is reduced or prevented. Isolated islet cells need not be cooled to inactivate enzymatic activity. Polystyrene sulfonate is a chymotrypsin inhibitor. It will protect islet cells from acinar proteolytic activity. It is expected that this will lead to improved islet yield while reducing mechanical, chemical and thermal trauma to islet cells. Collaginase enzyme can be inactivated by cooling to 4° C. It can be regenerated by treating gel with EDTA or sodium citrate. It can be sterilized and reused for most cost effective isolation of cells. Other miscellaneous uses may involve preparations of dental and cartilage molds which may help reduce inflammatory responses and scarring. Similarly, the material can be used to develop novel biomaterials such as sutures, tendons or hemodialysis fibers with improved biocompatibility and immune tolerance. In vitro, microhydrogel formulation can be used to prolong preservation of platelets, cells and organs and its in-vitro culture.

b. Treatment of Gonococcal sepsis: A number of pathogens, such as streptococcal pyrogenes and Neisseria gonococci evade the immune system by combining with Factor H. Factor H inactivates C3 convertase at the microbial surface and allows cells to multiply. This phenomenon is responsible for virulence and resistance to antibiotics resulting in severe sepsis and death of patients. Polystyrene sulfonate avidly binds to Factor H more than Factor D. Furthermore, such binding inactivates Factor H and activates the complement system. The hydrogel of polystyrene sulfonate of 200 kD pore size thus can readily be prepared. The size of the hydrogel can also be reduced to 1 micron or less to encapsulate noncellular material such as antibiotics. The use of such ultramicrohydrogel for parenteral therapy of gonoccocal sepsis, will lead to competitive binding with Factor H by polystyrene sulfonate and activate alternate complement pathways. Antibiotics that are also encapsulated then will provide bactericidal effects to exposed gonococci and cause improvement in clinical condition.

c. Treatment of HIV infection to prevent or halt progression to AIDS. GP120/41 viral receptor binds to CD4+T cells. This binding is critical for i) gaining entry into CD4 and dendritic cells, ii) multiplication of viruses inside such cells, iii) pathological destruction of CD4 and dendritic cells, and iv) destabilizing immune responses. Rapid viremia, destruction of immune cells leads to AIDS. Both gp 120 and 41 evade activation of complement system by binding to Factor H and thus obtain a foothold on CD4 and dendritic cells. Blockage of Factor H, for example, by Factor H antibodies have led to efficient killing of HIV viruses by complement activation in in-vitro experiments. Use of immunotoxins, such as cholera or diphtheria toxin is another approach pursued to prevent progression of HIV to AIDS. By depleting Factor H with polystyrene sulfonate and providing antiviral medicine as encapsulated therapy, a complement system can be activated and at the same time bring efficient killing of HIV viruses thus exposed.

Ultrapurified alginate and sulfonic polymer can thus be combined to develop controlled release drugs that may help activate the complement system and cause efficient removal of pathogens, such as gonococci, streptococcal pyrogenes and HIV viruses. Activation of complement system provides the necessary costimuli and cytokines to activate adaptive immunity. Therefore, the same strategy can be used for developing vaccines against pathogens. In addition to bacteria and viruses, tumors such as renal cell cancer, bladder cancer and cervical cancer also evade complement system detection by secreting Factor H-like substances and may be amenable for similar therapy. Preparedness against bio-terrorism and military preparedness for overseas posts are of growing national importance for the U.S.A. as well as several countries around the world. Encapsulation of CPG, ODN or endotoxin with DNA vaccines against potential bio-threat agents such as Ebola, anthrax, listeria and tularemia in a 200 kD pore size hydrogel, will help activate innate immune responses quickly and develop protective adaptive immunity faster with lessening of side effects.

In conclusion, the invention accomplishes the object of providing a means for immune switching in the form of a hydrogel containing an ultrapurified polymer having an endotoxin content below 700 E.U./g. The hydrogel may be applied in a variety of forms or be used to coat foreign materials or to encapsulate cellular or noncellular material. A hydrogel with pore size between 50 kDa and 100 kDa generally functions as a complement inhibitor while the embodiment having larger pores greater than 200 kDa generally functions as a complement activator.

It will be understood that certain features and subcombinations are of utility and may be employed without reference to other features and subcombinations. This is contemplated by and is within the scope of my claims. It is further obvious that various changes may be made in details within the scope of my claims without departing from the spirit of my invention. It is, therefore, to be understood that my invention is not to be limited to the specific details detailed shown and described.

What is claimed is:

1. A method of clinically treating a disease in a host, wherein said disease causes modulation of at least one of Factor D, Factor H and CD 4 in the host immune system, the method comprising the steps of:
providing polystyrene sulfonate;
coating the polystyrene sulfonate with a hydrogel to form an encapsulated polystyrene sulfonate, wherein the hydrogel includes a gelling agent for providing immunoisolatory functions to enable selective targeting of different immune diseases by the encapsulated polystyrene sulfonate; and
administering to the host an effective dose of the encapsulated polystyrene sulfonate for therapeutically and selectively modulating at least one of the Factor D, Factor H and CD 4 in the host immune system.

2. The method of claim 1, wherein the selective targeting comprises selective binding of the Factor D and selective exclusion of the Factor H by the encapsulated polystyrene sulfonate.

3. The method of claim 1, wherein the administering step further comprises contacting the encapsulated polystyrene sulfonate with at least one of blood, body fluid and mucous membranes of the host.

4. The method of claim 3, wherein the encapsulated polystyrene sulfonate comprises a formulation selected from the group consisting of powders, liquids, tablets, suppositories, injectable mediums, inhalation formulations, toothpastes, microcapsules and ex-vivo devices.

5. The method of claim 1, wherein the encapsulated polystyrene sulfonate is administered in combination with an immune modulating drug.

6. The method of claim 1, wherein the encapsulated polystyrene sulfonate is administered in combination with a chemotherapeutic agent.

7. The method of claim 1, wherein the encapsulated polystyrene sulfonate is administered in combination with a cytotoxic agent.

8. The method of claim 1, wherein the encapsulated polystyrene sulfonate is administered in combination with a vaccine.

9. The method of claim 1, wherein selective targeting of different immune diseases is enabled by selective binding of the Factor D by the encapsulated polystyrene sulfonate for treatment of complement activated diseases.

10. The method of claim 1, wherein selective targeting of different immune diseases is enabled by selective binding of the Factor H by the encapsulated polystyrene sulfonate for treatment of diseases that cause immune evasions of cytotoxic immune responses.

11. The method of claim 1, wherein the CD 4 is therapeutically modulated to inhibit maturation of dendritic cells for transplant tolerance.

12. The method of claim 9, wherein the complement activated diseases are of acute origin.

13. The method of claim 12, wherein the acute complement activated diseases comprise acute respiratory distress (ARDS), severe burns, multiple trauma, sepsis, acute stroke, coronary by-pass surgery, angioplasty, myocardial infarction, disseminated intravascular coagulopathy, inhalation anthrax, plague, tularemia induced sepsis due to bioterrorism, complications due to hemodialysis or cardiopulmonary bypass procedures in heart surgery, and transplantation rejection reactions due to xenotransplants, cell transplants and organ transplants.

14. The method of claim 10, wherein the diseases that cause immune evasion of cytotoxic immune responses comprise at least one of bacterial diseases, viral diseases, parasitic diseases and cancers.

15. The method of claim 1, wherein the Factor D, the Factor H and CD 4 are all therapeutically modulated for the therapy of drug-resistant bacterial infections, drug-resistant viral infections and drug-resistant cancer metastasis.

16. The method of claim 1, wherein the hydrogel comprises an ultrapurified alginate having an endotoxin content less than 700 E.U./g.

17. The method of claim 1, wherein the gelling agent provides immunoisolatory functions by enabling a pore size of the hydrogel to be adjusted.

18. The method of claim 1, wherein the gelling agent comprises CaCl.

19. The method of claim 1, wherein the Factor H has a molecular weight of 150 kD and the Factor D has a molecular weight of 25 kD.

20. The method of claim 17, further comprising the step of adjusting the pore size of the hydrogel to have a nominal molecular weight cutoff between 50 kD and 100 kD.

21. The method of claim 17, further comprising the step of adjusting the pore size of the hydrogel to have a nominal molecular weight cutoff of at least 200 kD.
